(19) United States
(12) Patent Application Publication
(10) Pub. No.: US 2022/0133600 A1
(43) Pub. Date: May 5, 2022

(54) System Having A Laser And An Adhesive For The Securing Of A Dental Restoration Part And Method For Securing A Restoration Part, In Particular A Dental Restoration Part

(71) Applicant: Ivoclar Vivadent AG, Schaan (LI)

(72) Inventors: Christian Niedrig, Rüthi (CH); Michael Barbisch, Klaus (AT); Thomas Köhler, Reichenau (DE)

(21) Appl. No.: 17/517,205

(22) Filed: Nov. 2, 2021

(30) Foreign Application Priority Data
Nov. 2, 2020 (EP) 20205294.0

(51) Int. Cl. A61K 6/30; A61C 13/10

(52) U.S. Cl. CPC A61K 6/30; A61C 13/10

(57) ABSTRACT

The invention relates to a system consisting of a laser which emits laser radiation with a wavelength of more than 1300 nm, in particular between 2750 nm and 3200 nm, and a dental adhesive for applying a dental restoration part to a pre-treated, in particular cleaned, adhesive base which is formed by hard dental tissue or by denture material. The viscous dental adhesive applied to the adhesive base, which is free of a dental restoration part, can be activated, in particular without blowing or agitation, by applying laser radiation.

### CROSS-REFERENCE TO RELATED APPLICATIONS

This application claims priority to European Patent Application No. 20205294.0 filed on Nov. 2, 2020, the disclosure of which is incorporated herein by reference in its entirety.

### TECHNICAL FIELD

The invention relates to a system comprising a laser and an adhesive, with the aid of which a dental restoration part can be bonded to hard dental tissue and a method for securing a restoration part, in particular a dental restoration part.

### BACKGROUND

The curing of dental materials with a manageable light curing device with multiple curing modes is known, for example, from WO 2016/202992 A1 and corresponding U.S. Pat. No. 10,376,350B2, which U.S. patent is hereby incorporated by reference in its entirety.

From US 2019/142700 A1, which is hereby incorporated by reference in its entirety, the application of photon energy is known, in order to energize dental materials to improve their physical handling properties, efficacy, deliverability, reactivity, polymerisation and/or mechanical post-curing properties, among other properties.

In order to adhere dental restoration parts, such as teeth, veneers, inlays, onlays or a prosthesis base on dental hard tissue (i.e. enamel and/or dentin) a special adhesive is used. An example of this is the adhesive “Adhese Universal” from the company Ivoclar Vivadent AG, for which processing instructions are available on the Internet.

However, in this case “restoration parts” are also to be understood to be restoration elements, which become restoration parts only as a result of curing, such as dental filling materials, composites, Alkasites, compomers, glass ionomer cements, etc.

Dental adhesives are self-curing and/or light-curing, presently light-curing in the vast majority of cases, having a sensitivity maximum in the range of visible light, namely around 470 nm, induced by the photoinitiator used, camphorquinone, but typically also with UV light components.

In order to achieve a successful adhesive bond, it is necessary to apply the adhesive carefully to the dental hard tissue. In order to achieve an effective bond, the adhesive must penetrate into the pores of the tooth substance.

Moreover, a uniform layer thickness is necessary. Typically, the layer thickness of the adhesive is less than 0.08 mm, preferably less than 0.03 mm, particularly preferably between 0.01 to 0.02 mm.

According to the processing instructions for use of the “Adhese Universal”, it is necessary for this purpose, to massage or stir the adhesive into the tooth substance using a small brush, a so-called “micro brush”. Such a processing step is also referred to as agitation.

The agitation must be performed for 15 to 30 seconds, e.g. 20 seconds.

According to the processing instructions, compressed air blowing is then performed in order to homogenise the layer thickness.

Several seconds are also to be allowed for this and compressed air must also be available at the location where the processing work takes place.

The method is comparatively cumbersome and time-consuming. Nevertheless, this multi-step and time-consuming method has been used for decades with different adhesives.

### SUMMARY

Therefore, the object of the invention is to create a system having a laser and an adhesive according to the claims, and a method for securing a restoration part, in particular a dental restoration part, according to the claims, which on the one hand is improved with regard to the process flow and is more universally applicable, and on the other hand is improved with regard to acceptance by both dentists and their patients.

In accordance with the invention, this object is achieved by the independent claims. Advantageous developments are apparent from the dependent claims.

In accordance with the invention, provision is made, prior to the application of the dental restoration part, in an additional step, to subject the adhesive previously applied on an adhesive base to a laser treatment. According to the invention, the adhesive used can be, for example, the “Adhese Universal” already mentioned, but also any other suitable dental adhesive. Preferably, the adhesive contains mixtures of different methacrylates in solvents (e.g. ethanol, acetone). The monomers may contain one or more methacrylate groups and further functional groups. Further functional groups serve to provide special product properties, such as carboxylic acid or phosphoric acid ester groups of self-etching and adhesion to hard dental tissue. The adhesives may additionally contain processing aids (e.g. rheology additives to achieve the typical viscosity of dental adhesives), stabilizers (radical scavengers), as well as photoinitiators and possibly parts of redox initiator systems for light and/or dual curing and/or self-curing.

The adhesive base can be a dental hard tissue, such as dentin or enamel, but e.g. also a dental restoration part, such as an artificial tooth, e.g. consisting of ceramic, or a prosthesis base, e.g. consisting of PMMA. Other dental restorations such as a crown, a partial crown, an inlay or an onlay, e.g. made of glass ceramic, oxide ceramic or CAD composite as an adhesive base, are also possible.

The system including a laser and an adhesive according to the invention can be used inside or outside the mouth of a patient. Preferably, it is used inside the patient's mouth.

Surprisingly, a considerable time savings can be achieved by virtue of the measure of applying irradiation additionally by means of an irradiation laser at a comparatively high wavelength in the infrared range, such as around 1500 nm, 1580 nm, around 3000 nm, or a wavelength between 1500 nm and 3000 nm. This is surprising, as it would be expected that an additional step would prolong the procedural process.

The adhesive applied to the adhesive base is preconditioned or activated by exposure to laser radiation. This activation leads to an improvement in the adhesion, and thus to an improvement in the shear strength of the cementation of the restoration that is later applied to the tooth.

By exposure to laser irradiation according to the invention, i.e. activation, the adhesive is brought into a state in which it is ready for immediate application of the dental restoration. According to the invention, this makes the step of agitating the applied adhesive, which takes at least 20 seconds, and the blowing step superfluous. Especially when the adhesive is applied in the patient's mouth, the patient is not inconvenienced by the long agitation time.

In this respect, the level of acceptance is increased, in particular if the step of blowing is also dispensed with. Especially sensitive patients whose dental nerves are possibly irritated often find the intensive feed of cold air to be unpleasant.

Moreover, the turbulent incident flow or overflow over water-containing soft tissue or liquids as a result of aerosols or droplets is considered to be risky especially during periods of an epidemic because they increase the possibility of infection.

In accordance with the invention, the laser emitting the laser radiation, the treatment laser, can be designed in a manner known per se either as a laser with a mains connection or power connection or optionally as one operated by a rechargeable battery. A bacteria removal laser can also be used as a treatment laser, in particular one according to EP 3 628 266 A1 and corresponding US 20200101315, which U.S. published application is hereby incorporated by reference in its entirety.

In a further embodiment, a light source is used in addition to the treatment laser. These two radiation sources preferably have different power levels. The treatment laser with the first power level is used to activate the adhesive applied to the adhesive base before the dental restoration is applied.

In a further step, the dental restoration part is applied and the adhesive, which is now between the adhesive base and the dental restoration part, is cured by the light source, i.e. the second radiation source with the second power level. With a direct restoration, the dental restoration part as a composite is applied, especially sculptable or flowable.

With an indirect application of the dental restoration part, a cement is applied between the adhesive applied to the adhesive base and/or to the restoration, in particular a crown, and the restoration.

Here it is advantageous if the power level of the second radiation source is below the first power level and the second radiation source emits a wavelength suitable for the polymerisation of the adhesive.

In a further step, in particular after application of a cement, the dental restoration part is applied and the adhesive, which is now located between the adhesive base and the dental restoration part, is cured by the light source, i.e. the second radiation source with the second power level. It is advantageous if the second radiation source emits a wavelength suitable for polymerization of the adhesive. The power level of a conventional dental light curing unit is e.g. 1200-3000 mW/cm².

Er:YAG solid state lasers are generally designed as floor-mounted apparatuses and have a mains connection or power connection. However, rollers are typically mounted on the floor-mounted apparatus so that the laser is movable and in this respect the mobility is improved in comparison with a (generally stationary) compressed air source, as it is required for blowing. Moreover, Er:YAG lasers of a few cubic decimetres in size have now been developed, which further benefits the mobility. Also, development in terms of size reduction continues unabated.

The system according to the invention having a laser and an adhesive is also more independent than the previously known systems, as it is not dependent on a compressed air source if the blowing step is omitted in its application.

However, in a modified embodiment of the invention it is not precluded that short additional blowing is effected, either before or after the third step, i.e. the laser treatment step.

Whilst specialist literature has referred for decades to how important agitation and blowing are for achieving the required penetration depth of the dental adhesive and homogenisation of the layer thickness, surprisingly it is demonstrated in accordance with the invention that the shear strength of a dental restoration applied in accordance with the invention can be even higher than in the case of a reference restoration which has been applied in a conventional manner, i.e. by means of agitation and blowing.

Preferably, the shear strength values, i.e. the shear bond strength values, are above the following limits:

The shear strength of the adhesive, such as “Adhese Universal” in self-etch mode, on a bovine substrate should be at least 25 MPa for dentin and at least 17 MPa for enamel. In this case, an adhesive in “self-etch mode” means that it contains a primer, a methacrylate monomer and other polymeric components with an acidic pH that is able to demineralise enamel and dentin while penetrating these conditioned surfaces.

In the case of an inventive dental restoration applied to tooth enamel, it was possible to achieve a shear strength of more than 22 MPa with an irradiation time of the adhesive treatment laser of 1 second.

In the case of a dental restoration applied to dentin, it was possible to achieve a shear strength of more than 34 MPa with an irradiation time of the treatment laser according to the invention of 5 seconds.

The dental restorations applied with the system according to the invention thus show high shear strengths, and this despite a shortened process sequence compared to the prior art.

In both cases, the strength was as good as or better than in the case of a dental restoration applied as a reference sample by agitation and blowing of the adhesive (22 MPa and 33 MPa respectively).

Specifically when applied to tooth enamel as an adhesive base, a significant time saving of about 20 seconds is thus achieved for each dental restoration.

In the system according to the invention comprising a laser and an adhesive for attaching a restoration part, namely a dental restoration part, it is envisaged that the adhesive base, for example the tooth substance, is first pre-treated in a pre-treatment step. This can be done by mechanically removing carious tooth structure, followed by a water spray application to clean dentin/smear layer/enamel material.

However, this can also include an etching step, e.g. etching with phosphoric acid, which, however, can be omitted when using universal adhesives.

In one advantageous embodiment of the invention, additional laser application by means of the treatment laser with an emission wavelength range of more than 1300 nm can also be used prior to application of the adhesive.

The adhesive is applied in a manner known per se in an application step, preferably by means of a professional applicator, such as a micro brush.

In the third step, the laser treatment step, according to the invention, the applied adhesive is subjected to a laser treatment with laser radiation with an emission maximum of, for example, between 1400 nm and 1600 nm or between about 1400 nm to about 1600 nm or of about 2940 nm. Water absorption bands exist at these wavelengths.

Surprisingly, this laser treatment of the—as it were raw—adhesive layer results in a conditioning or activation of the adhesive, which leads to a significantly increased shear strength compared to the process without this step. It thus emerges that without blowing or agitating, a surprisingly high shear strength can be achieved only by using the combination of a treatment laser with an adhesive according to the invention. The time-consuming blowing and agitation can be omitted according to the invention.

A possible explanation for this surprising effect is that the treatment laser removes the water content and/or highly volatile organic components from the tubules of the dentin, so that the adhesive can penetrate the tubules or diffuse into them better, thus improving retention.

As far as tooth enamel is concerned, a further possible explanation is that the temperature increase, which occurs in accordance with the invention when using the laser through the entire adhesive results in a stronger etching effect of the acid monomer.

In an advantageous embodiment, both the agitation step and the blowing step can thus be omitted when using the system according to the invention. However, even if these two additional steps are performed, as is the case according to the classical method for securing dental restorations, the system according to the invention consisting of a laser and an adhesive results in a substantially good or equally good or even better shear strength.

In another application step, the dental restoration part is applied to the—conditioned or activated adhesive and is pressed thereon in a manner known per se.

In a manner likewise known per se, polymerisation or curing of the adhesive by means of the second radiation source is then performed; light polymerisation is typically performed at a wavelength between 400 and 480 nm. This ensures secure fixing of the dental restoration.

In a manner known per se, dental adhesives are also suitable for providing hydrophilic adhesive bonds, i.e. for coupling both to dentin and tooth enamel. It is known that dentin has a slightly higher water proportion than tooth enamel, of e.g. about 10%. This is possibly the explanation for the results of a series of tests carried out in connection with the system consisting of a laser and an adhesive according to the invention, from which the result emerges that, in the case of dentin, a longer irradiation time with the treatment laser than with enamel tends to lead to the desired high shear strength.

It is understood that the optimum irradiation time can be adapted within wide ranges to the requirements.

Even in the case of a longer irradiation time of 5 seconds of the adhesive with the treatment laser in accordance with the invention, a significant time savings is achieved in comparison with the standard agitation time of 20 to 30 seconds.

For adhesive joints involving both dentin and enamel, it is preferable to use a longer adhesive irradiation time of, for example, 5 seconds, which is optimal for dentin and allows only a little less shear strength for enamel.

According to the invention, the laser of the system of the invention comprising a laser and an adhesive emits radiation in a wave range between 1300 nm and 4500 nm. The radiation from the laser is directed to the exposed adhesive applied to the adhesive base. The radiation penetrates the adhesive and enters the adhesive base.

Preferably, a control device is provided for this treatment laser by means of which an irradiation time can be adjusted or is fixed. The radiation of the treatment laser conditions the adhesive applied to the adhesive base in such a way that it leads to a high shear strength of the adhesive bond when used for the securing of dental restorations. Examples include but are not limited to, a central processing unit (CPU), a programmable logic controller (PLC), a PID controller, and/or other units with computing power.

It is understood that for this purpose there should be no objects reducing the irradiation between the radiation emitting surface of the treatment laser and the surface of the adhesive, such as an applied dental restoration.

In the system according to the invention, it is consequently provided that the treatment laser is directly applied to the exposed adhesive.

Preferably, the light guide rod is attached to a handpiece that either contains the radiation source of the treatment laser or is connected via a flexible light guide to a standing device that contains the radiation source.

For the application of the adhesive treatment radiation according to the invention, the handpiece is preferably brought by the dentist into a position in which the radiation emitting surface is located above the adhesive, preferably in such a position that the radiation emitting surface extends parallel or substantially parallel to the adhesive layer.

Even if, according to the invention, a solid-state laser is preferably used, which also preferably operates in pulsed mode, it is also possible to use a less expensive diode laser instead, which preferably operates without pulsed mode, i.e. continuously. The diode laser can also emit treatment laser radiation continuously. With such a diode laser, it is possible to exploit the absorption maximum of water, which is between 1400 nm and 1600 nm. Compared to an Er:YAG laser, a diode laser has the advantage that it is much more compact and therefore easier to handle.

It also has a longer lifetime, often more than 30,000 hours, than other laser types, such as an Er:YAG laser.

Diode lasers are also very energy efficient and have a comparatively high electrical/optical efficiency of 25 to over 50%. Furthermore, their low power degradation of less than 1%/1000 h when operating at nominal current is also an enormous advantage over other laser types.

In one embodiment of the system according to the invention, a diode laser is used as the laser, which emits in the range of 1400 nm to 1600 nm, i.e. in a range in which absorption bands of water are present.

It is preferred to provide a system having a laser and a dental adhesive, wherein the laser emits laser radiation with a wavelength of more than 1300 nm, wherein the dental adhesive is adapted for application to a pre-treated adhesive base formed by dental hard tissue, denture material or dental restorative material, wherein the dental adhesive is adapted for treatment by applying laser radiation, and wherein the dental restoration part is secured on the treated dental adhesive.

It is preferred that the wavelength is in the range of 2750 nm and 3200 nm, wherein the pre-treated adhesive base is cleaned, wherein the dental adhesive is adapted for treatment by activation without blowing or agitation, wherein the laser radiation is applied while the dental adhesive is free of a dental restoration part, and wherein the dental restoration part is secured on the activated dental adhesive.

It is preferred that the laser as a treatment laser is a first radiation source, and wherein a second radiation source is designed as an LED light source or as a laser, and the dental adhesive is adapted for curing by the second radiation source with a second power level, which second power level is below a first power level of the treatment laser and comprises a wavelength configured for polymerisation.

It is preferred that the laser is designed as a solid-state laser with at least one emission maximum at a wavelength between 2750 nm and 3200 nm, and is designed as a pulsed laser comprising an Er:YAG laser.

It is preferred that during the application of laser radiation to provide a laser treatment, the laser treatment takes place for a time in a range of 0.5 sec to 15 sec, or in a range of 2 sec to 8 sec, and emits pulsed laser radiation with a pulse/pause ratio in a range of 1:1 to 1:1000 or in a range of 1:5 to 1:200 or provides a continuously emitting laser radiation.

It is preferred that the laser is configured as an unpulsed laser comprising a diode laser.

It is preferred that the laser is configured with at least one emission maximum at a wavelength between about 1400 and 1600 nm.

It is preferred that the laser is used is a bacteria removal laser.

It is preferred that the laser emits radiation into a light guide, the distal end of which is bent and/or flexible, and wherein the radiation of the laser can be directed through the light guide to the dental adhesive and the adhesive base.

It is preferred that a sensor is provided which responds to radiation in the wavelength range between 1000 nm and 15000 nm and has a sensitivity maximum above or below the emission maximum of the laser.

It is preferred that the sensor is configured as a reflection detection sensor which is attached to a handpiece of the laser or proximate thereto and is movable together therewith.

It is preferred that the sensor detects radiation emitted by the laser when directing the radiation of the laser towards the adhesive base and the dental adhesive.

It is preferred that a target or aiming light source is mounted adjacent to and substantially parallel to the laser and that the target or aiming light source emits visible light on a focus spot.

It is preferred that the laser includes a deflection tip with which outgoing radiation is deflected with respect to an axis of the laser.

It is preferred that during the application of laser radiation to provide a laser treatment, the laser treatment takes place for a time in the range of 0.5 s to 15 s, or 2 sec to 8 sec, and emits pulsed laser radiation with a pulse/pause ratio in a range of 1:1 to 1:1000 or 1:5 to 1:200 or at about 1:25.

It is preferred that a method is provided for securing a restoration part having a dental restoration part using a dental adhesive to secure the part onto an adhesive base including the steps of pre-treating the adhesive base wherein pre-treating includes cleaning the adhesive base, applying the dental adhesive to the adhesive base, treating the dental adhesive applied to the adhesive base with a laser which emits radiation in a wavelength range of 1300 nm or more or 1400 nm or more, and applying the restoration part onto the dental adhesive and adhesive base after treatment of the dental adhesive.

It is preferred that the laser is designed as a solid-state laser having at least one emission maximum at a wavelength between 2750 nm and 3200 nm, and comprising an Er:YAG laser.

It is preferred that the laser is configured with at least one emission relative maximum at a wavelength between about 1400 and 1600 nm, and wherein the laser is configured as a diode laser.

It is preferred that during the laser treatment step, the laser treatment is effected for a time in the range of 0.5 s to 15 s, or a range of 2 s to 8 s, and wherein the laser is configured as a pulsed laser or as a continuously emitting laser, wherein the pulsed laser outputs pulsed laser radiation at a pulse/pause ratio in a range of 1:1 to 1:1000 or a range of 1:5 to 1:200 or at about 1:25.

It is preferred that in the step of applying the dental adhesive, the dental adhesive is applied in a layer thickness in a range of 0.03 mm to 0.1 mm or 0.01 mm to 0.02 mm.

It is preferred that the laser treatment step directly follows the step of applying the dental adhesive without any massaging-in and/or agitation of the dental adhesive into or onto the adhesive base and without any blowing of the dental adhesive.

It is preferred that the laser comprises a bacteria removal laser.

It is preferred that the laser outputs radiation into a light guide, the distal end of which is offset and/or flexible, and wherein the radiation of the laser is directed by the light guide to the dental adhesive and the adhesive base.

It is preferred that with the pre-treatment step or before or after the pre-treatment step, the adhesive base is irradiated, roughened and/or heated, with the laser.

### BRIEF DESCRIPTION OF THE DRAWINGS

Further advantages, details and features will be apparent from the following description of several embodiments of the invention with reference to the drawings.

In the drawings:

FIG. 1 shows a schematic perspective view of a system including a laser and adhesive, in one embodiment of the invention, to explain the system including a laser and an adhesive in accordance with the invention;

FIG. 2 shows a detail of a further embodiment of an inventive system including a solid-state laser and an adhesive;

FIG. 3 shows a schematic view of the adhesive curing after application of the dental restoration part to the adhesive;

FIG. 4 shows a graph to illustrate the shear strength which can be achieved at different irradiation times during the formation of the adhesive base as enamel as well as when using a pulsed laser;

FIG. 5 shows a graph to illustrate the shear strength which can be achieved at different irradiation times during the formation of the adhesive base as dentin and using a pulsed laser;

FIG. 6 shows a graph to illustrate the shear strength which can be achieved at different irradiation times during the formation of the adhesive base as enamel and using a non-pulsed laser;

FIG. 7 shows a graph to illustrate the shear strength which can be achieved at different irradiation times during the formation of the adhesive base as dentin and using a non-pulsed laser;

FIG. 8a shows a further embodiment of a treatment laser having a tip which is particularly suitable for performing the laser treatment step in deep cavities; and

FIG. 8b shows a further embodiment of a treatment laser having a tip which is particularly suitable for performing the laser treatment step in deep cavities.

### DETAILED DESCRIPTION

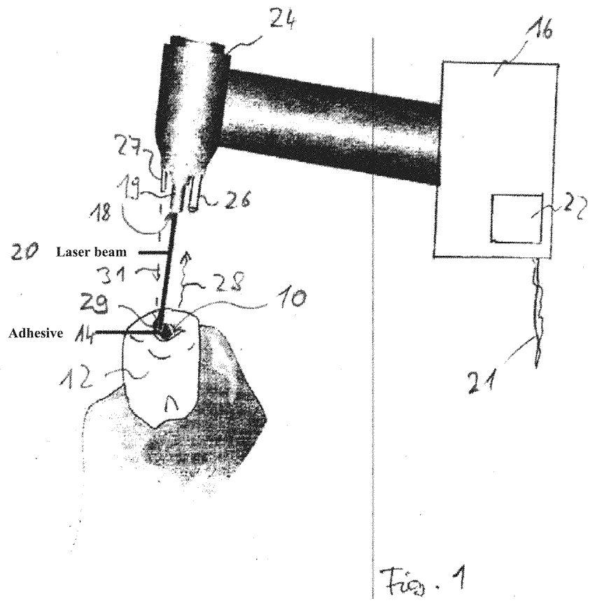

An embodiment of a system in accordance with the invention is described with reference to FIG. 1.

FIG. 1 shows an adhesive base 10 on or at a tooth 12, in the illustrated exemplified embodiment a molar or a premolar with a cavity in the occlusal surface.

For pre-treatment purposes, milling, grinding and/or polishing of the tooth hard tissue is performed in a manner known per se until only healthy tissue is present.

If required, an etchant can be applied in a manner known per se for pre-treatment purposes.

In a manner likewise known per se, an adhesive 14 is applied to the adhesive base 10.

By means of a spreading apparatus, such as e.g., by means of a micro brush or another suitable dental applicator, the applied adhesive 14 is distributed to produce a layer of less than 0.03 mm.

This application constitutes the application step, the second step.

In a third step, in accordance with the invention, the laser treatment step, a treatment laser 16 is oriented with its radiation-emitting surface 18 on a light guide rod as a possible exemplified embodiment of a light guide 19 such that the exiting laser beam which forms the radiation 20 is directed onto the adhesive layer 14. The laser 16 is switched on and as a result the adhesive layer is subjected to a laser treatment.

In the embodiment shown, the laser 16 is a solid-state laser, namely an Er:YAG laser. It has an emission maximum at a wavelength of 2940 nm and a mains or power connection 21. Preferably, the Er:YAG laser has an electrical power of between 0.4 watts and 10 watts, preferably between 1 watt and 2 watts and particularly preferably of 1.3 watts.

In the illustrated exemplified embodiment, the adhesive base 10 consists mostly of tooth enamel and to a lesser extent of dentin. It is important that the adhesive 14 demonstrates good shear strength throughout.

For example, an irradiation time of 5 seconds is specified by a control device 22, and accordingly the laser 16, which may be designed as a pulsed (tests 1 and 2) or continuous (tests 3 and 4) laser, is switched on for 5 seconds.

By this laser exposure with the laser radiation 20 in the wavelength range mentioned, the adhesive 14 is conditioned in such a way that it has a high shear strength after application of the dental restoration and after polymerisation of the adhesive, i.e. after completion. This system consisting of treatment laser and adhesive according to the invention is the only way to achieve the desired surprisingly high shear strength in a time-saving manner.

In the first two embodiment examples (tests 1 and 2), a pulsed laser with a pump current of 300 amperes with a pulse duration of 300 microseconds, a pulse frequency of 150 Hz and an average laser power, i.e. averaged over the pulse sequence, of 1.3 W is used. The beam diameter is 5.4 mm.

A total of four tests were carried out with embodiments using the system consisting of a laser and an adhesive according to the invention.

In the first test, bovine tooth enamel was selected as the adhesive base 10. The test was repeated at different irradiation times by the pulsed laser and the following shear strength values were achieved according to table 1 below (pursuant to ISO 29022):

TABLE 1
(Test 1)

| | | | | | | | |
|---|---|---|---|---|---|---|---|
| Radiation/s | 10 | 7.5 | 5 | 2.5 | 1 | 0 | n/a |
| | | | | | | | (Reference |
| | | | | | | | according to |
| | | | | | | | standard) |
| Number of tests | 5 | 5 | 20 | 5 | 5 | 4 | 5 |
| Average shear | 18.38 | 21.75 | 20.22 | 21.47 | 22.94 | 19.11 | 22.09 |
| strength value | | | | | | | |
| in MPa | | | | | | | |
| Standard | 2.64 | 7.69 | 4.84 | 2.46 | 6.57 | 1.31 | 2.67 |
| deviation | | | | | | | |
| Min | 14.93 | 15.16 | 11.57 | 17.8 | 19.25 | 17.83 | 19.64 |
| Max | 21.3 | 32.81 | 31.45 | 23.62 | 34.61 | 20.71 | 25.56 |
| Fracture | adhesive | adhesive | adhesive | adhesive | adhesive | adhesive | adhesive |
| appearance | | | | | | | |

The fracture pattern can be adhesive or cohesive, as shown in the table. An adhesive fracture is a fracture that runs exactly along the phase interface between the surface of the part to be joined and the adhesive. This type of fracture results in a complete separation of the adhesive from the substrate surface(s). The test was repeated with bovine dentin as the adhesive base 10.

A cohesive fracture means that the adhesion of the adhesive at the phase boundaries is greater than the fracture strength of the adhesive base. The adhesive joint therefore survives the fracture test and the substrate breaks instead.

The shear strength values shown in table 2 below were achieved (pursuant to ISO 29022):

TABLE 2
(Test 2)

| | | | | | | | |
|---|---|---|---|---|---|---|---|
| Radiation/s | 10 | 7.5 | 5 | 2.5 | 1 | 0 | n/a |
| | | | | | | | (Reference |
| | | | | | | | according |
| | | | | | | | to standard) |
| Number of | 5 | 5 | 20 | 5 | 5 | 5 | 5 |
| tests | | | | | | | |
| Average shear | 29.27 | 31.21 | 34.19 | 30.80 | 17.89 | 18.47 | 33.08 |
| strength value | | | | | | | |
| in MPa | | | | | | | |
| Standard | 5.18 | 3.65 | 7.59 | 2.07 | 3.36 | 1.92 | 3.89 |
| deviation | | | | | | | |
| Min | 22.93 | 26.98 | 20.46 | 27.59 | 13.31 | 16.18 | 26.69 |
| Max | 36.72 | 36.15 | 46.56 | 32.97 | 22.10 | 21.38 | 37.31 |
| Fracture | 3/5 | 4/5 | 19/20 | cohesive | adhesive | adhesive | cohesive |
| appearance | cohesive | cohesive | cohesive | | | | |

Tests 1 and 2 were carried out using pulsed laser radiation and the system consisting of a laser and an adhesive according to the invention.

Experiments 3 and 4 were also carried out using a continuously operated, i.e. unpulsed, diode laser. The results are shown in the tables below.

In Test 3, bovine enamel was again chosen as the adhesive base 10. The test was carried out with different irradiation times of the continuously operated diode laser. The data obtained with Er:YAG laser irradiation in pulse mode are also given in Table 3 for reference purposes.

Furthermore, the values obtained without laser irradiation are also given and, in addition, reference values are also given which are obtained when working according to the standard procedure with blowing and agitation. The following shear strength values were obtained, according to Table 3 below (according to ISO 29022):

TABLE 3
(Test 3)

| | 5 s 1 W | 1 s 5 W | | | with blowing and agitation |
|---|---|---|---|---|---|
| | continuous | continuous | 5 s 1.3 W | without | (reference values according |
| Irradiation-parameter | diode laser | diode laser | Er:YAG | irradiation | to standard) |
| Number of tests | 5 | 5 | 20 | 4 | 5 |
| Average shear | 22.60 | 24.14 | 20.22 | 19.11 | 22.09 |
| strength value in MPa | | | | | |
| Standard deviation | 4.86 | 2.97 | 4.84 | 1.31 | 2.67 |
| Min | 17.6 | 19.88 | 11.57 | 17.83 | 19.64 |
| Max | 28.83 | 27.21 | 31.45 | 20.71 | 25.56 |
| Fracture appearance | adhesive | adhesive | adhesive | adhesive | adhesive |

Test 4 was carried out with bovine dentin as the bonding base 10, i.e. as test 2, but using a continuously operated diode laser.

The shear strength values (according to ISO 29022) were obtained as shown in table 4 below:

TABLE 4
(Test 4)

| | 5 s 1 W | 1 s 5.1 W | | | with fading and agitating |
|---|---|---|---|---|---|
| | continuous | continuous | 5 s 1.3 W | Without | (reference values according |
| Irradiation-parameter | diode laser | diode laser | Er:YAG | irradiation | to standard) |
| Number of attempts | 5 | 4 | 20 | 5 | 5 |
| Mean shear | 29.32 | 28.16 | 34.19 | 18.47 | 33.08 |
| strength in MPa | | | | | |
| Standard deviation | 2.76 | 6.44 | 7.59 | 1.92 | 3.89 |
| Min | 24.99 | 22.38 | 20.46 | 16.18 | 26.69 |
| Max | 31.83 | 37.04 | 46.56 | 21.38 | 37.31 |
| Fracture appearance | cohesive | cohesive | 19/20 | cohesive | cohesive |
| | | | cohesive | | |

Through these two tests using a continuously operated diode laser (tests 3 and 4), it could be shown that the timesaving use of the system of laser and adhesive according to the invention is not limited to the use of pulsed laser radiation and does not come at the expense of shear strength.

Furthermore, the results of the tests according to the invention with both the continuously operated diode laser and the pulsed laser support the fact that a similarly high or even partly higher shear strength is achieved according to the invention than with the standard procedure by agitation and blowing.

The effect according to the invention can be explained by the fact that the treatment laser removes the water content and/or highly volatile organic components from the tubules of the dentin or the pores of the enamel. The space created in the tubules or pores now allows the adhesive to penetrate the tubules or diffuse into them more easily. As a result of this penetration of the adhesive, the retention and thus also the shear strength is increased.

Tooth hard tissue has different reflection properties than an applied adhesive 14. In order to make it easier to orient the hand piece 24 of the laser 16, a sensor 26 is provided on the hand piece 24 adjacent the radiation-emitting surface 18 and detects the reflected radiation 28 and feeds same to the control apparatus 22.

Preferably, a signal can thus be indicated which signalises the correct orientation of the hand piece 24.

The hand piece 24 also has a target light source which is designed preferably as a target laser 27 and emits visible light e.g. in the blue, green, yellow or red wavelength range. White or other mixed light is also possible. The target laser 27 is physically connected to the treatment laser 16. It is oriented such that its target beam 31 impinges upon a focus spot 29 of the treatment laser 16. At this location, it generates a visible light point. It is thus oriented axially parallel or substantially axially parallel thereto.

However, the axis of the target laser 27 can also be inclined slightly with respect to the axis of the treatment laser 16, as illustrated in FIG. 1.

By means of the target laser 27, the orientation of the treatment laser 16 can be controlled and optionally readjusted. It is switched on preferably before the laser 16 is switched on. The hand piece 24 is oriented to the desired position and then the treatment laser 16 is switched on.

The target laser can also be integrated in the laser housing 16 and can couple the visible light directly into the optical waveguide 19 of the laser such that no “dedicated” radiation guidance is necessary. This has the advantage that the target light uses the exact same optics and therefore (apart from any chromatic aberration) represents the actually illuminated surface quite effectively—irrespective of the distance to the treatment surface and any trigonometric considerations in different exit openings.

In this respect, reference is made to the typical design of laser systems with an integrated target laser. However, a laser does not have to be used as the target light source because coherence is not required in this case, only visible illumination. Therefore, in principle every light source which outputs visible light is suitable for generating the target beam.

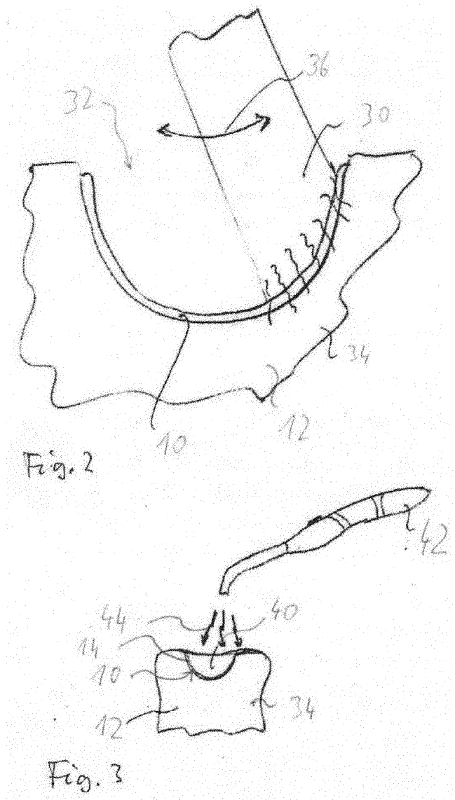

FIG. 2 shows a further embodiment of a system in accordance with the invention in an enlarged detail. The radiation 20 output by the laser 16 impinges in the form of the radiation 30 onto the surface of the adhesive 14 which is applied in the cavity 32. The radiation 30 passes through the adhesive layer 14 and also penetrates into the tooth hard tissue 34.

In order to permit the best possible irradiation, the hand piece 24 is pivoted such that the radiation 30 impinges according to the arrow 36 as perpendicularly as possible onto the surface of the adhesive layer 14. The surface of the adhesive 14 is exposed and is thus freely accessible for the radiation 30 because a dental restoration part 40, see FIG. 3, is not yet applied.

FIG. 3 shows the state in which a dental restoration part 40 is already introduced into the cavity 32, the dental restoration part 40 has thus been applied to the adhesive layer 14.

The adhesive 14 is polymerised via a hand-held light curing apparatus 42. The output polymerisation radiation 44 passes for this purpose through the dental restoration part 40 which is permeable to laser radiation 44 in the range of visible light.

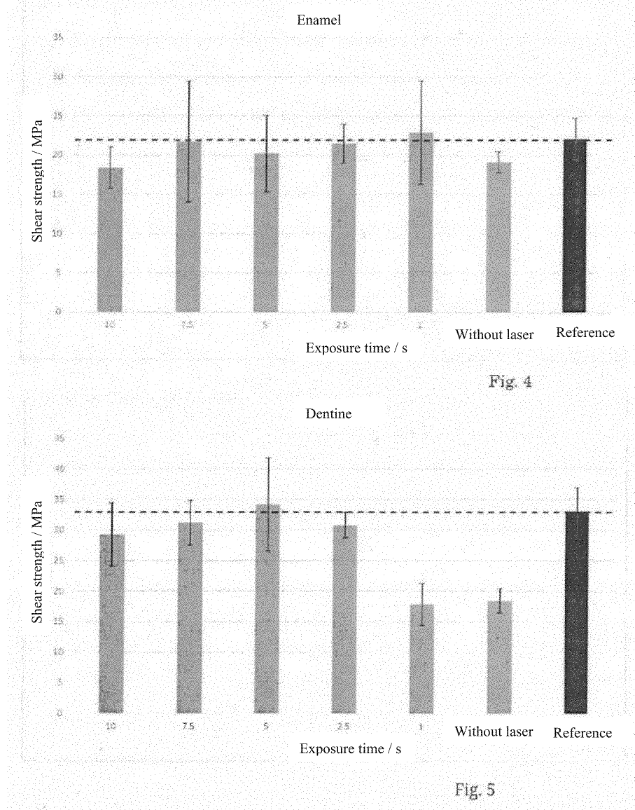

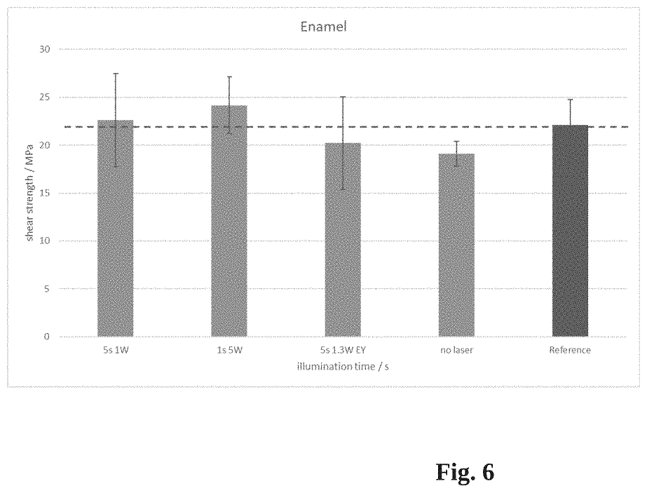

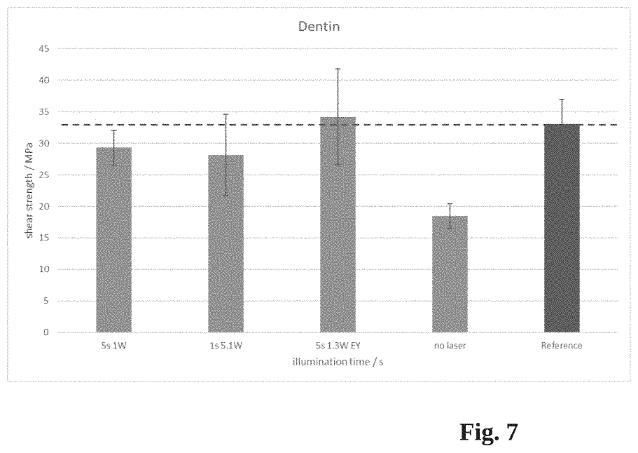

FIG. 4 shows a graph plotted based on the values according to Table 1 and FIG. 5 shows a graph plotted based on the values of Table 2. FIG. 6 shows a diagram plotted based on the values according to Table 3 and FIG. 7 shows a diagram plotted based on the values according to Table 4.

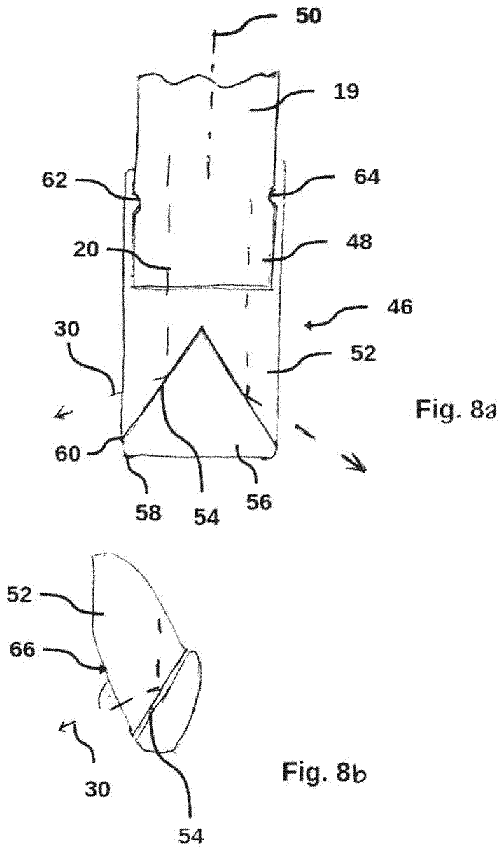

In a modified embodiment shown in FIG. 8a, the treatment laser 16 is provided with a deflection tip 46. In this embodiment, the deflection tip 46 is fitted onto the distal end 48 of the light guide 19. Alternatively, the deflection tip 46 can also be formed in one piece with the light guide 19, i.e. integrated therein.

The deflection tip 46 is arranged coaxially with respect to an axis 50 of the light guide 19. It consists of a body 52 made of a material which is transparent for the radiation 20. At its distal end, the deflection tip 46 has an internal cone 54. This has a reflective coating applied thereto or is configured in another suitable manner so that it reflects impinging radiation 20.

The radiation 20 is thereby deflected from the axis 50 and emerges laterally from the deflection tip 46 as radiation 30. It is intended to strike the adhesive 14 applied to the adhesive base 10 and act there as treatment radiation 30.

This embodiment is particularly suitable for the treatment of deep cavities which are provided with the adhesive 14. The treatment radiation 30 is scattered and deflected all around such that treatment radiation 30 is applied uniformly even to oblique surfaces of the adhesive (cf. FIG. 2).

The internal cone 54 is provided with a suitable stopper 56 consisting of a soft material which preferably has a rounded portion 58 formed in this case as an end radius. The rounded portion projects with respect to a sharp circumferential end edge 60 of the body 52 and in this respect covers same. The stopper 56 is adhered in the internal cone 54. The projecting rounded portion 58 prevents damage to the laser 16 and injury to the patient.

The internal cone 54 can have a cone angle of 90 degrees. Then, the radiation 30 exits the body 52 perpendicularly with respect to the axis 50. In the illustrated embodiment, the cone angle of the internal cone 54 is or is about 65 degrees. Accordingly, the radiation 30 exits the deflection tip 46 slightly obliquely forwards. In the case of this solution, the deflection tip 46 does not have to be introduced so deeply into a deep cavity.

It is also possible to apply a reflective coating only partially to the internal cone 46 such that some of the radiation, e.g. 50% is reflected and the remainder is allowed to pass through. In the case of this embodiment, the stopper 56 is likewise permeable to the radiation.

In the case of this solution, the output radiation then impinges not only upon the side walls but also upon the base of the cavity, which is favourable in many cases.

By virtue of the fact that the deflection tip 46 is detachable, it is also possible to alternate between different emission characteristics.

In this case, the deflection tip 46 is fitted onto the distal end 48 of the light guide 19. For this purpose, it has an annular groove 62 which is intended to ensure that an annular web 64 of the body 52 engages therein and supports the deflection tip 46 without any clearance on the light guide 19. The body 52 consists of an optionally slightly elastic, radiation-permeable material such that, when the body of the deflection tip 46 is slid onto the light guide 19 the annular web 64 latches elastically into the annular groove.

Alternatively, it is possible to provide at this location a sleeve fabricated of an elastic synthetic material which engages over both the body 52 and also the end 48 of the light guide 19.

In the case of these two embodiments, the deflection tip 46 is held captively on the light guide 19 but can be removed with manual force or optionally with the aid of a suitable tool, such as mini-pliers.

In the case of a cone angle which is below 90 degrees, the body 52 of the deflection tip 46 can also be provided with an external cone 66 at its distal end, as shown in FIG. 8b.

FIG. 8b shows a section of a detail relating to FIG. 8a. The external cone 66 is adapted in terms of its cone angle to the cone angle of the internal cone 54. The radiation 30 leaves the body 52 through the external cone 66 in a direction perpendicular to the external cone 66 so as to avoid reflection losses.

The terms “about” and “substantially” are intended to include the degree of error or uncertainty associated with measurement of the particular quantity or shape as one of ordinary skill in the art would understand.

1. A system comprising
a laser and a dental adhesive,
wherein the laser emits laser radiation with a wavelength of 1300 nm or more,
wherein the dental adhesive is suitable for application to a pre-treated adhesive base formed by dental hard tissue, denture material or dental restorative material,
wherein the dental adhesive is adapted for treatment by applying laser radiation, and
wherein the dental restoration part is secured on the treated dental adhesive.

2. The system according to claim 1,
wherein the wavelength is in the range of 2750 nm and 3200 nm,
wherein the pre-treated adhesive base is cleaned,
wherein the dental adhesive is adapted for treatment by activation without blowing or agitation,
wherein the laser radiation is applied while the dental adhesive is free of a dental restoration part, and
wherein the dental restoration part is secured on the activated dental adhesive.

3. The system according to claim 1,
wherein the laser (16) is designed as a solid-state laser with at least one emission maximum at a wavelength between 2750 nm and 3200 nm, and is designed as a pulsed laser comprising an Er:YAG laser.

4. The system according to claim 1,
wherein, during the application of laser radiation to provide a laser treatment, the laser treatment takes place for a time in a range of 0.5 sec to 15 sec or in a range of 2 sec to 8 sec, and
emits pulsed laser radiation (20) with a pulse/pause ratio in a range of 1:1 to 1:1000 or in a range of 1:5 to 1:200 or 1:25 or provides a continuously emitting laser radiation.

5. The system according to claim 1,
wherein the laser (16) is configured as an unpulsed laser comprising a diode laser.

6. The system according to claim 1,
wherein the laser (16) is configured with at least one emission maximum at a wavelength between about 1400 and 1600 nm.

7. The system according to claim 1,
wherein the adhesive (14) is applied in a layer thickness of 0.08 or less mm, or 0.03 mm or less and or between 0.01 to 0.02 mm.

8. The system as claimed in claim 1,
wherein the laser (16) is used is a bacteria removal laser.

9. The system according to claim 1,
wherein the laser (16) emits radiation (20) into a light guide (19), the distal end of which is bent and/or flexible, and
wherein the radiation (20) of the laser (16) can be directed through the light guide (19) to the dental adhesive (14) and the adhesive base (10).

10. The system according to claim 1,
wherein a sensor (26) is provided which responds to radiation in the wavelength range between 1000 nm and 15000 nm and has a sensitivity maximum above or below the emission maximum of the laser.

11. The system according to claim 10,
wherein the sensor (26) is configured as a reflection detection sensor which is attached to a handpiece (24) of the laser (16) or proximate thereto and is movable together therewith.

12. The system according to claim 11,
wherein the sensor (26) detects radiation emitted by the laser (16) when directing the radiation of the laser (16) towards the adhesive base (10) and the dental adhesive (14).

13. The system according to claim 10
wherein a target or aiming light source (27) is mounted adjacent to and substantially parallel to the laser (16).

14. The system according to claim 13,
wherein the target or aiming light source (27) emits light visible on a focus spot (29).

15. The system according to claim 1,
wherein the laser (16) comprises a deflection tip (46) with which outgoing radiation (20) is deflected with respect to an axis (50) of the laser (16).

16. The system according to claim 5,
wherein, during the application of laser radiation to provide a laser treatment, the laser treatment takes place for a time in the range of 2 sec to 8 sec, and emits pulsed laser radiation (20) with a pulse/pause ratio in a range of 1:5 to 1:200 or at about 1:25.

17. A method for securing a restoration part comprising a dental restoration part (40) using a dental adhesive (14) to secure the part onto an adhesive base (10), comprising pre-treating the adhesive base (10) wherein pre-treating includes cleaning the adhesive base (10),
applying the dental adhesive (14) to the adhesive base (10),
treating the dental adhesive (14) applied to the adhesive base (10) with a laser which emits radiation (20) in a wavelength range of 1300 nm or more or 1400 nm or more, and
applying the restoration part (40) onto the dental adhesive (14) and adhesive base (10).

18. The method as claimed in claim 17,
wherein the laser (16) is designed as a solid-state laser having at least one emission maximum at a wavelength between 2750 nm and 3200 nm, and comprising an Er:YAG laser.

19. The method as claimed in claim 17,
wherein the laser (16) is configured with at least one emission relative maximum at a wavelength between about 1400 and 1600 nm, and
wherein the laser (16) is configured as a diode laser.

20. The method as claimed in claim 17,
wherein, during the laser treatment step, the laser treatment is effected for a time in the range of 0.5 s to 15 s, or 2 s to 8 s, and
wherein the laser is configured as a pulsed laser or as a continuously emitting laser,
wherein the pulsed laser outputs pulsed laser radiation (20) at a pulse/pause ratio in the range of 1:1 to 1:1000 or of 1:5 to 1:200 or at about 1:25.

21. The method as claimed in claim 17,
wherein, in the step of applying the dental adhesive, the dental adhesive (14) is applied in a layer thickness in a range of 0.03 mm to 0.1 mm.

22. The method as claimed in claim 17,
wherein, in the step of applying the dental adhesive, the dental adhesive (14) is applied in a layer thickness in a range of 0.01 mm to 0.02 mm.

23. The method as claimed in claim 17,
wherein the laser treatment step directly follows the step of applying the dental adhesive (14), without any massaging-in and/or agitation of the dental adhesive (14) into or onto the adhesive base (10), and without any blowing of the dental adhesive (14).

24. The method as claimed in claim 17,
wherein the laser (16) comprises a bacteria removal laser.

25. The method as claimed in claim 17,
wherein the laser (16) outputs radiation (20) into a light guide (19), the distal end of which is offset and/or flexible, and
wherein the radiation (20) of the laser (16) is directed by the light guide (19) to the dental adhesive (14) and the adhesive base (10).

26. The method as claimed in claim 17,
wherein, with the pre-treatment step or before or after the pre-treatment step, the adhesive base (10) is irradiated, roughened and/or heated, with the laser (16).
